(19) United States
(12) Patent Application Publication
(10) Pub. No.: US 2022/0296514 A1
(43) Pub. Date: Sep. 22, 2022

(54) Composition and Method of Preparation for Lipid Formulations Comprising Charged Lipids

(71) Applicant: FormuRx Pharmaceuticals Co., Ltd., Taipei (TW)

(72) Inventors: Chin-tin Chen, Taipei (TW); Chia Ying Lee, New Taipei City (TW); Tsuimin Tsai, Taipei (TW); Po-Chun Peng, Taipei (TW)

(21) Appl. No.: 17/697,939

(22) Filed: Mar. 18, 2022

Related U.S. Application Data
(60) Provisional application No. 63/163,005, filed on Mar. 18, 2021.

(51) Int. Cl. A61K 9/107; A61K 9/127; A61K 31/704; A61K 31/65; A61K 9/51; A61K 33/243

(52) U.S. Cl. CPC A61K 9/1075; A61K 9/127; A61K 31/704; A61K 31/65; A61K 9/1277; A61K 9/5123; A61K 9/5192; A61K 33/243

(57) ABSTRACT

A lipid-based nanoparticle (LNP) with high DL ratio and normalized release. The LNP of the present invention comprises an outer lipid monolayer encapsulating a plurality of lipid-active pharmaceutical ingredient (API)-ion complexes, wherein each lipid-API-ion complex comprises a complex of anionic lipid, API and ion wherein the API comprises a positively charged form of an API and wherein the outer lipid monolayer of the LNP comprises neutral lipids. The present invention further comprises a method of preparation of the LNP of the present invention.

### CROSS REFERENCE TO RELATED APPLICATIONS

The present application claims priority to U.S. Provisional Application No. 63/163,005, filed on Mar. 18, 2021, entitled “Lipid Based Nanoparticle Delivery System for Hydrophilic Charged Compound.”

### FIELD OF THE INVENTION

The present invention relates to lipid-based nanoparticles (LNP) with high DL ratio and normalized release, method of preparation of the LNP and uses thereof.

### BACKGROUND OF THE INVENTION

Lipid-based nanoparticles (LNP) have become important vehicles for delivery of various therapeutic compounds to targeted sites since LNPs provide advantages over other drug delivery means due to better biodistribution and toxicity mitigation. Specifically, the small size of LNPs better avoids removal from the bloodstream by the reticuloendothelial system so that they can circulate for a longer period of time in a patient. In addition, LNPs are able to better target diseased sites such as cancer tumors and inflammation sites by extravasation through leaky vasculature of the cancer tumor and disease inflamed regions.

However, to provide even more effective therapy using LNPs, there is a need to substantially increase the drug to lipid ratio (D/L ratio) of LNPs so that more of the therapeutic compounds can be carried per LNP. In addition, there is a need to improve the ability of the LNP to target disease site by increasing release rate at the targeted disease site while maintaining stability elsewhere.

The use of rapid mixing in conjunction with electrostatic forces between components comprising the LNP can substantially increase the D/L ratio as well as increase capability to target diseased sites while maintaining stability in non-targeted sites in a patient. Specifically, the use of rapid mixing to more effectively mix negatively electrostatically charged lipids and positively electrostatically charged API can substantially increase D/L ratio. Furthermore, the net negative electrostatic charge environment of acidic cancer tumor regions helps to increase the release rate of the LNP at the target disease site by disrupting the structure of LNP that comprises charged components such as charged lipids and active pharmaceutical ingredients.

An objective of the present invention comprises substantially raising drug to lipid ratio of the LNP of the present invention compared to those of existing LNPs. Another objective of the invention comprises improving stability as well as the ability for the LNP to target the diseased site, thus improving therapeutic effects thereof.

### BRIEF DESCRIPTION OF THE DRAWINGS

FIG. 1 illustrates an embodiment of the LNP 1 of the present invention.

FIG. 2 illustrates a method of preparation 100 for the LNP 1 of the present invention.

FIG. 3 illustrates various factors that can affect properties of the LNP 1 of the present invention.

FIG. 4 illustrates the zeta potential of LNP dox 1 of the present invention prepared using the method of preparation 100 of the present invention with various dialysis buffers 252.

FIG. 5 illustrates in vitro drug release of Lipo-Dox and LNP dox 1 of the present invention prepared using the method of preparation 100 of the present invention with various dialysis buffers 252.

FIG. 6 illustrates normalized release of LNP dox 1 of the present invention prepared using the method of preparation 100 of the present invention with different lipid compositions.

FIG. 7 illustrates particle size distribution of LNP dox 1 of the present invention prepared using the method of preparation 100 of the present invention with various lipid compositions.

FIG. 8A summarizes the formulation of LNP dox 1 of the present invention. FIG. 8B illustrates various characteristics of LNP dox 1 of the present invention with the formulation summarized in FIG. 8A.

FIGS. 9A and 9B illustrate therapeutic effect of LNP dox 1 of the present invention as compared to Doxil®, with FIG. 9A showing change in tumor volume over time and FIG. 9B showing percent survival over time.

FIG. 10 illustrates an exemplary embodiment of a rapid mixing system 200.

FIG. 11 illustrates an exemplary embodiment of microfluidic device 300 for performing rapid mixing.

FIG. 12 shows LNP 1 of the present invention entering lysosomes of tumor cells via endocytosis.

### DETAILED DESCRIPTION OF THE PRESENT INVENTION

The compositions of the present invention can comprise, consist of, or consist essentially of the essential elements and limitations of the invention described herein, as well as any of the additional or optional ingredients, components, or limitations described herein.

As used in the specification and claims, the singular form “a” “an” and “the” include plural references unless the context clearly dictates otherwise. For example, the term “a” cell includes a plurality of cells, including mixtures thereof.

“About” in the context of amount values refers to an average deviation of maximum ±20%, preferably ±10% or more preferably ±5% based on the indicated value. For example, an amount of about 30 mol % anionic lipid refers to 30 mol %±6 mol %, preferably 30 mol %±3 mol % or more preferably 30 mol %±1.5 mol % anionic lipid with respect to the total lipid/amphiphile molarity.

“Lipid” refers to its conventional sense as a generic term encompassing fats, lipids, and alcohol-ether soluble constituents of protoplasm, which are insoluble in water. Lipids are composed of fats, fatty oils, essential oils, waxes, steroid, sterols, phospholipids, glycolipids, sulfolipids, aminolipids, chromolipids, and fatty acids. The term encompasses both naturally occurring and synthetic lipids. Preferred lipids in connection with the present invention are: steroids and sterol, particularly cholesterol, phospholipids, including phosphatidyl and phosphatidylcholines and phosphatidylethanolamines, and sphingomyelins. Where there are fatty acids, they could be about 12-24 carbon chains in length, containing up to 6 double bonds. The fatty acids are linked to the backbone, which may be derived from glycerol. The fatty acids within one lipid can be different (asymmetric), or there may be only 1 fatty acid chain present, e.g., lysolecithins. Mixed formulations are also possible, particularly when the non-cationic lipids are derived from natural sources, such as lecithins (phosphatidylcholines) purified from egg yolk, bovine heart, brain, or liver, or soybean.

“Neutral lipids” are lipids which have a neutral net charge. “Anionic lipids” are lipid molecules which have a negative net charge. These can be selected from sterols or lipids such as cholesterol, phospholipids, lysolipids, lysophospholipids, sphingolipids or pegylated lipids with a negative net charge. “Amphiphilic lipids” are lipid molecules which exhibit both hydrophilic and hydrophobic properties. These can be selected from sterols or lipids such as cholesterol, phospholipids, lysolipids, lysophospholipids, sphingolipids or pegylated lipids with amphiphilic properties, which may have a negative, neutral, or positive net charge. Useful neutral and anionic lipids thereby include: phosphatidylserines, phosphatidylglycerols, phosphatidylinositols (not limited to a specific sugar), fatty acids, sterols containing a carboxylic acid group for example, cholesterol, phosphatidylethanolamines (PE) such as 1,2-diacyl-sn-glycero-3-phosphoethanolamines including, but not limited to 1,2-dioleoylphosphoethanolamine (DOPE), 1,2-distearoylphosphoethanolamine (DSPE), or 1,2-dihexadecoylphosphoethanolamine (DHPE), phosphatidylcholines (PC) such as 1,2-diacyl-glycero-3-phosphocholines including, but not limited to 1,2-distearoylphosphocholine (DSPC), 1,2-dipalmitoylphosphocholine (DPPC), 1,2-dimyristoylphosphocholine (DMPC), egg PC or soybean PC and sphingomyelins. The fatty acids linked to the glycerol backbone are not limited to a specific length or number of double bonds. Phospholipids may also have two different fatty acids.

An “effective amount” is an amount sufficient to effect beneficial or desired results.

An effective amount can be administered in one or more administrations, applications or dosages.

A “subject,” “individual” or “patient” is used interchangeably herein, which refers to a vertebrate, preferably a mammal, more preferably a human. Mammals include, but are not limited to, murines, simians, humans, farm animals, sport animals, and pets.

The “polydispersity index” (PDI) is defined as a measure of the heterogeneity of sizes of molecules or particles in a mixture such as a mixture of LNPs calculated as square of the standard deviation of the size distribution divided by the square of the mean of the size distribution.

The “drug to lipid ratio” (D/L ratio) is defined as the weight of drug (mg) divided by the weight of phospholipid (mg) in the lipid nanoparticle.

The “normalized release” is defined as release ratio in pH 5.4 environment divided by release ratio in pH 7.4 environment wherein the release ratio is defined as the percentage of API released from LNP. A high normalized release is a result of high release ratio in pH 5.4 environment as compared to the release ratio in pH 7.4 environment. Since tumor sites present acidic environment, a high normalized release is a metric indicating tumor targeting capability of an LNP.

The “rapid mixing” as defined by Djamel Ghernaout and Heinrich Roder articles^{1,2}, hereby incorporated in their entirety, is mixing that provides quick and efficient dispersion of two or more fluids to achieve complete mixing of the two or more fluids including mixing of various components comprising each fluid. Various types of rapid mixing have been developed. These include mechanical mixing, which is conventionally used for coagulant mixing in water treatment, diffusion mixing by a pressured water jet, in-line static mixing, in-line mechanical mixing, hydraulic mixing and mechanical flash mixing. ¹ Djamel Ghernaout and Ahmed Boucherit (2015), Journal of Research & Developments in Chemistry, DOI: 10.5171/2015.926518, page. 22.² Heinrich Order, Kosuke Make, Hong Cheng and M. C. Ramachandra Shastry, Methods 34(2004) 15-27

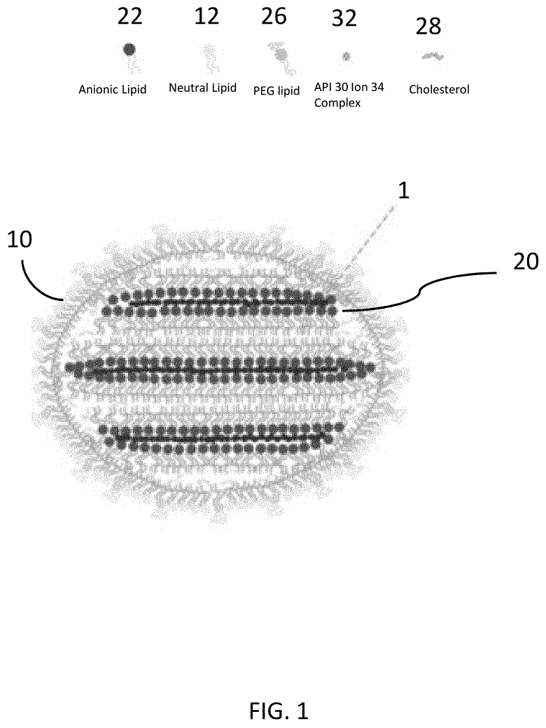

FIG. 1 illustrates an embodiment of the LNP 1 of the present invention. As illustrated in FIG. 1, an embodiment of the LNP 1 of the present invention comprises a lipid monolayer 10 encapsulating a plurality of lipid-API-ion complexes 20 wherein each lipid-API-ion complex 20 comprises a complex 20 of charged lipid 22, charged active pharmaceutical ingredient (API) 30 and ion 34 wherein the charged lipid 22 and ion 34 both have an opposite charge from the charge of the API 30. For example, if the API 30 has a positive charge, then both the charged lipid 22 and ion 34 are both anionic, and if the API 30 has a negative charge, then both the charged lipid 22 and ion 34 are both cationic.

In an embodiment, the lipid monolayer 10 of the LNP 1 of the present invention preferably comprises neutral lipids 12 such as but not limited to L-α-phosphatidylcholine, hydrogenated soybean phosphatidylcholine (HSPC), 1,2-dioeoyl-sn-glycero-3-phosphocholine (DOPC), 1,2-dioleoylphosphoethanolamine (DOPE), 1,2-distearoylphosphoethanolamine (DSPE), and/or polyethyleneglycol-derivated distearoylphosphatidylethanolamine (PEG-DSPE), or 1,2-dihexadecoylphosphoethanolamine (DHPE), phosphatidylcholine (PC) such as 1,2-diacyl-glycero-3-phosphocholines including but not limited to 1,2-distearoylphosphocholine (DSPC), 1,2-dipalmitoylphosphocholine (DPPC), and 1,2-dimyristoyl-sn-glycero-3-phosphocholine (DMPC). In an embodiment, the lipid monolayer 10 of the LNP 1 of the present invention preferably comprises less than 20%, 15%, 10%, 5% or 1% of positively or negatively charged lipids.

In an embodiment, the charged lipid 22 of the lipid-API-ion complex 22 preferably comprises anionic lipids 22 which may comprise phosphatidylglycerol, cardioplipin, diacylphosphatidylserine, diacylphosphatidic acid, lysylphosphatidylglycerol, egg L-α-phosphatidylglycerol (EPG), 1,2-dimyristoyl-sn-glycero-3-phosphoglycerol (DMPG), 1,2-dipalmitoyl-sn-glycero-3-phosphoglycerol (DPPG), 1,2-dioleoyl-sn-glycero-3-phospho-(1′-rac-glycerol) (DOPG), 1,2-dioeoyl-sn-glycero-3-phosphate (DOPA), 1,2-dioleoyl-sn-glycero-3-phospho-L-serine (DOPS), and other anionic modifying groups joined to neutral lipids. In another embodiment, the charged lipid 22 does not comprise any phosphatidyl glycerol or any derivatives thereof. In an embodiment, the LNP 1 of the present invention does not comprise any phosphatidyl glycerol or any derivatives thereof. The charge on each anionic lipid 22 may be in the range of about −1 to −5 eV, about −1 to −3 eV, about −1 to −2 eV. In an embodiment, the lipid-API-ion complexes 20 of the LNP 1 of the present invention preferably each comprises less than 20%, 15%, 10%, 5% or 1% of neutral lipids 12.

The lipid monolayer 10 of the LNP 1 of the present invention may further comprise neutral lipids with PEG 26 such as DSPE-PEG2000 and/or DMG-PEG. In an embodiment, the PEG comprises in the range of about 1% to about 10% by weight of the neutral lipids 12. The LNP 1 and the lipid-API-ion complexes 20 may each further comprise steroids such as cholesterol, cholestanol, lanosterol, and the like. In an embodiment, the lipid monolayer 10 of the LNP 1 comprises more than 60%, 80%, 90%, 95% or 100% neutral lipids 12. In another embodiment, the lipid content of the lipid-API-ion complex 20 comprises more than 60%, 80%, 90%, 95% or 100% anionic lipids 22.

In an embodiment, the API 30 comprises a positively charged and hydrophilic compound. In another embodiment, the API 30 comprises a positively charged and hydrophilic antineoplastic drug. In another embodiment, the API 30 comprises positively charged forms of doxorubicin (dox), cisplatin (cDDP) or doxycycline (doxy). In an embodiment, the API 30 obtains the positive charge by hydration process. In an embodiment, doxorubicin, cisplatin, or doxycycline obtains positive charge by means of hydration as known in the art. In an embodiment, cisplatin may be hydrated for about 10 min to about 180 min, about 20 min to about 150 min, about 30 min to about 120 min, about 40 min to about 90 min, and about 50 min to about 70 min. In an embodiment, doxorubicin or doxycycline may be hydrated for about 30 sec to about 50 min, about 1 min to about 30 min, about 5 min to about 20 min, or about 7 min to about 15 min. In another embodiment, the lipid-API-ion complexes 20 may further comprise adjuvants, or one or more additional APIs 30, etc. . . . .

In an embodiment, the ion 34 comprises an ion with opposite charge to that of the charged API 30 and is capable of complexing with the charged API 30 and anionic lipid 22. In an embodiment, the ion 34 may comprise counter ion of the dialysis buffer 252 such as sulfate from magnesium sulfate or sodium sulfate. In another embodiment, the ion 34 comprises citrate, acetate or phosphate. In an embodiment, the lipid-API-ion complex 20 comprises the charged lipid 22 surrounding the API 30 and ion 34. In an embodiment, the lipid-API-ion complex 20 comprises the charged lipid 22 encapsulating the API 30 and ion 34. In an embodiment, the lipid-API-ion complex 20 comprises a micelle comprising a lipid monolayer encapsulating a complex 32 of the API 30 and the ion 34 wherein the lipid monolayer comprises the charged lipid 22.

The drug to lipid ratio for prior art LNP encapsulating cisplatin is from about 0.014 to about 0.1.³ In contrast, using the formulation and method of preparation disclosed in the Example 3 below, the drug to lipid ratio of the LNP encapsulating cisplatin of the present invention is substantially higher at from about 0.12 to about 0.2 such as about 0.12, about 0.13, about 0.14, about 0.15, about 0.16, about 0.17, about 0.18, about 0.19 or about 0.2 including all numbers ranges and numbers falling within these values. In addition, the drug to lipid ratio for prior art LNP encapsulating doxy is about 0.06.⁴ In contrast, using the formulation and method of preparation disclosed in the Example 3 below, the drug to lipid ratio of the LNP encapsulating doxy of the present invention is substantially higher at about 0.1 to about 0.5 such as about 0.1, about 0.15, about 0.2, about 0.25, about 0.3, about 0.35, about 0.4, about 0.45 or about 0.5 including all numbers ranges and numbers falling within these values. In addition, the drug to lipid ratio for prior art LNP encapsulating dox is from about 0.125.⁵ In contrast, using the formulation and method of preparation disclosed in the Example 3 below, the drug to lipid ratio of the LNP encapsulating dox of the present invention is substantially higher at about 0.15 to about 0.35 such as about 0.15, about 0.175, about 0.2, about 0.225, about 0.25, about 0.3 or about 0.35 including all numbers ranges and numbers falling within these values. ³ http://www.hoajonline.com/journals/pdf/2052-9341-4-2.pdf⁴ Budai, M., et al., Liposomal oxytetracycline and doxycycline: studies on enhancement of encapsulation efficiency. Drug Discov Ther, 2009. 3(1): p. 13-7.⁵ Doxil® Nordström, Rickard et al. “Quantitative Cryo-TEM Reveals New Structural Details of Doxil-Like PEGylated Liposomal Doxorubicin Formulation.” Pharmaceutics vol. 13, 1 123. 19 Jan. 2021, doi:10.3390/pharmaceutics13010123

In an embodiment, the LNP 1 of the present invention does not comprise a therapeutically effective amount of nucleic acids, nucleotides or polynucleotides including RNA or RNA-based or RNA derived API such as siRNA, micro RNA, antisense oligonucleotides, ribozymes, plasmids and/or immune stimulating nucleic acids. In another embodiment, the LNP 1 of the present invention comprises less than 5%, 2%, 1%, 0.1%, 0.001% by molar ratio of nucleic acids, nucleotides or polynucleotides including RNA or RNA-based or RNA derived API siRNA, micro RNA, antisense oligonucleotides, ribozymes, plasmids and/or immune stimulating nucleic acids. In another embodiment, the LNP 1 of the present invention is completely free of nucleic acids, nucleotides or polynucleotides including RNA or RNA-based or RNA derived API siRNA, micro RNA, antisense oligonucleotides, ribozymes, plasmids and/or immune stimulating nucleic acids.

It is well known in the art that LNPs with 200 nm or smaller diameter are able to better avoid removal by the reticuloendothelial system than larger sized particles resulting in circulation in a subject's bloodstream for a far longer period of time than larger sized LNPs. Furthermore, LNPs with diameter smaller than 100 nm are able to preferentially accumulate at disease sites such as tumors and sites of infection and inflammation due to their ability to extravasate through leaky vasculature in such regions due to the EPR effect as discussed in further detail below.

Therefore, in an embodiment, diameter of the LNP 1 of the present invention encapsulating cDDP is from about 25 nm to about 200 nm such as about 25 nm, about 50 nm, about 75 nm, about 100 nm, about 125 nm, about 150 nm, about 175 nm or about 200 nm including all numbers ranges and numbers falling within these values. In an embodiment, diameter of the LNP 1 of the present invention encapsulating dox is from about 25 nm to about 200 nm such as about 25 nm, about 50 nm, about 75 nm, about 100 nm, about 125 nm, about 150 nm, about 175 nm or about 200 nm including all numbers ranges and numbers falling within these values. In an embodiment, diameter of the LNP 1 of the present invention encapsulating doxy is from about 5 to about 300 nm such as about 25 nm, about 50 nm, about 100 nm, about 150 nm, about 200 nm, about 250 nm or about 300 nm including all numbers ranges and numbers falling within these values.

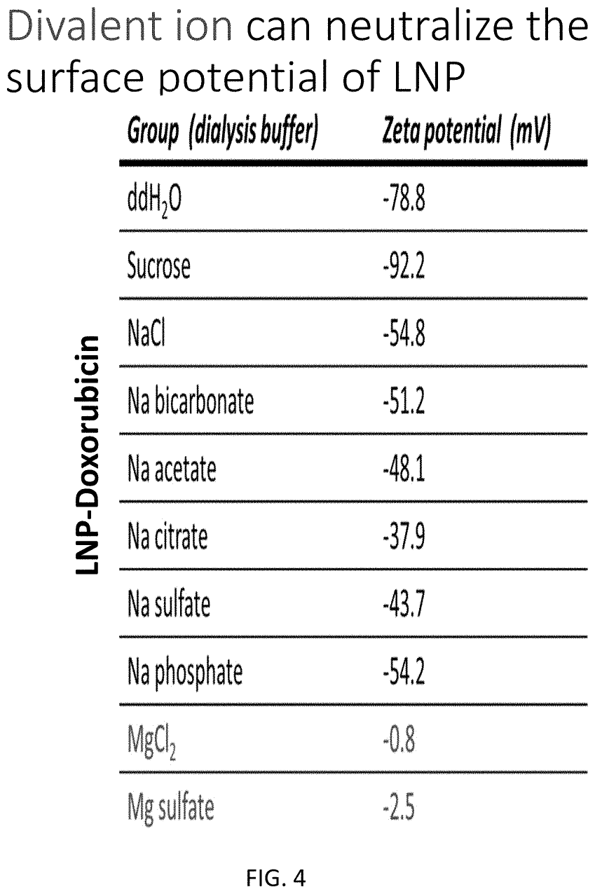

It is also known in the art that charged particles are easily removed from blood circulation by the reticuloendothelial system. The LNP 1 of the present invention better avoids this removal mechanism by having an overall near neutral or neutral electrostatic charge as the positive charge of the API 30 balances out with the negative charge of the anionic lipid 22 as well as that of the charged ions 34 of the lipid-API-ion complex 20. Zeta potentials are shown in FIG. 4 for LNP made with method of preparation 100 of the present invention using various dialysis buffers 252. Overall charge of LNP 1 of the present invention is measured by dynamic light scattering using Nanoparticle analyzer SZ-100 (HORIBA, Kyoto, Japan) in which charge is indicated by the amount of charged lipid affecting light scattering. Therefore, in an embodiment, the overall charge of the LNP of the present invention may range from about ±100, about ±75, about ±50, about ±40, about ±30, about ±20, about ±10, about ±5, about ±2 or about ±1 zeta value.

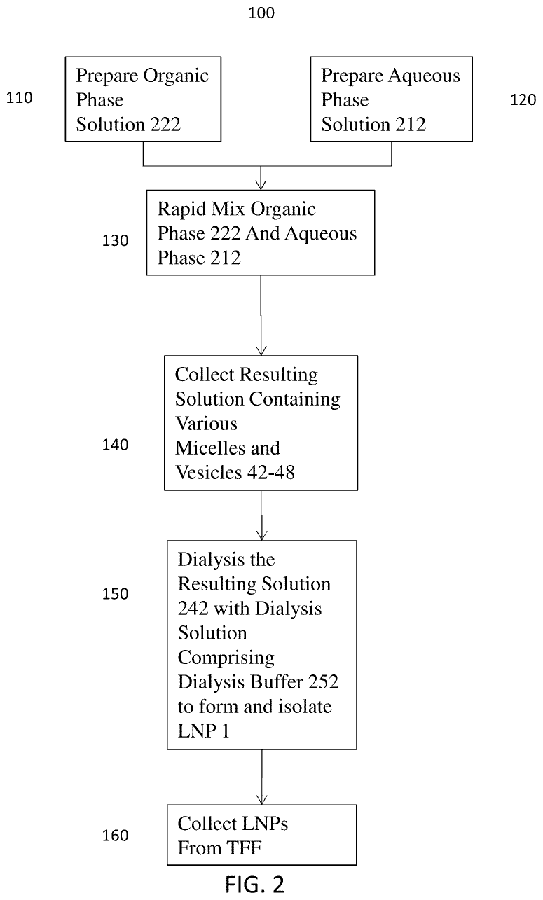

The present invention also comprises a method for preparing the LNP 1 of the present invention. An embodiment of the method of preparation 100 of the present invention is illustrated in FIG. 2. The method of preparation 100 of the present invention utilizes rapid mixing to amplify the effect of electrostatic attraction between charged components comprising the LNP 1 of the present invention. In addition, the method of preparation 100 of the present invention uses dialysis buffer 252 during dialysis process to facilitate formation and isolation of the LNP 1 of the present invention as illustrated in FIG. 1.

In an embodiment, the rapid mixing comprises mixing aqueous phase 212 and organic phase 222 wherein the organic phase 222 comprises both neutral lipids 12 as well as anionic lipids 22 dissolved in a solvent such as ethanol and the aqueous phase 212 comprises positively charged API 30 such as positively charged dox, doxy or cDDP dissolved in a solvent such as NaCl. In an embodiment, rapid mixing mixes anionic lipids 22 of organic phase 222 with cationic API 30 of aqueous phase 212 in such a way as to quickly and efficiently disperse the anionic lipids 22 and cationic API 30 to increase the effect of electrostatic attraction of the two components, raising D/L ratio of the LNP 1 of the present invention. Rapid mixing can be distinguished from passive mixing. Specifically, whereas passive mixing comprises mixing that passively occurs by merely combining two or more fluids together such as by injection of two or more fluids into a receptacle, rapid mixing comprises not only combining two or more fluids but also actively mixing two or more fluids with a mixing element 230 to quickly and uniformly disperse the fluids including their components. In another embodiment, rapid mixing comprises not only combining two or more fluids but also mixing the two or more fluids with a mixing element in conjunction with fluid pumps as described in further detail below. In an embodiment, the rapid mixing achieves uniform dispersion of the fluids and their components being mixed within about 100 microseconds to about 2 seconds such as about 100 microsecond, about 500 microsecond, about 1 milliseconds, about 50 milliseconds, about 100 milliseconds, about 200 milliseconds, about 500 milliseconds, about 800 milliseconds, about 1 second about 2 seconds including all numbers ranges and numbers falling within these values.

In an embodiment, cholesterol of the organic phase 222 can be replaced with nonamphiphilic fat such as medium chain triglyceride (MCT). In another embodiment, the solvent in which cholesterol is dissolved may be selected from methanol, isopropanol, and other water soluble organic solvents. In another embodiment, the solvent in which the positively charged API 30 is dissolved may be PBS, ddH₂O, sucrose or dextrose.

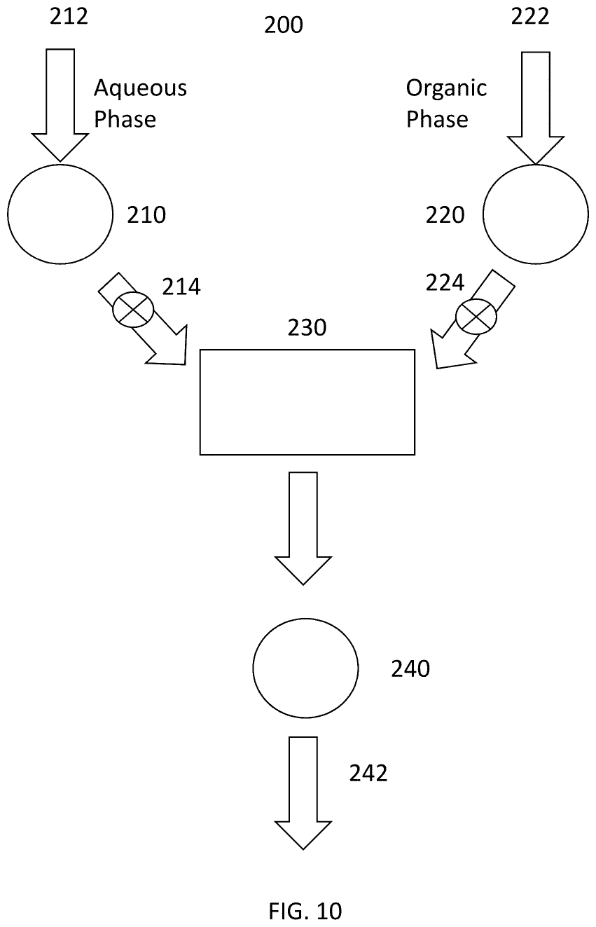

FIG. 10 illustrates an exemplary embodiment of rapid mixing system 200. As shown in FIG. 10, the rapid mixing system 200 comprises an aqueous phase receptacle 210, an aqueous phase pump 214, an organic phase receptacle 220, an organic phase pump 224, a mixing element 230 and resulting solution receptacle 240. The aqueous phase receptacle 210 is configured to receive the aqueous phase 212, and the organic phase receptacle 220 is configured to receive the organic phase 222. Aqueous phase pump 214 and organic phase pump 224 are each configured to pump the aqueous phase 212 and organic phase 224 to the mixing element 230 for mixing, respectively. Preferably, the aqueous phase pump 214 and organic phase pump 224 may each be independently controlled to provide the desired flow volume and/or flow rate for each phase 212 and 222. In an embodiment, the mixing element 230 is configured to rapid mix the aqueous phase 212 and organic phase 222. In another embodiment, the mixing element 230 is configured to rapid mix the aqueous phase 212 and organic phase 222 in conjunction with pumping action of the aqueous phase pump 214 and organic phase pump 224. In an embodiment, the mixing element 230 comprises mixing elements used in the art for rapid mixing fluids capable of achieving the desired result of quickly dispersing anionic lipid 22 and positively charged API 30 uniformly to increase the effect of electrostatic attraction of those charged components. There are various mixing elements 230 capable of rapid mixing anionic lipid 22 and positively charged API 30 in a way that substantially increases the effect of electrostatic attraction of those components above and beyond what is possible by mere passive mixing as discussed in Ghernaout and Roder. For example, in an embodiment, the mixing element 230 may be as simple as a T or Y junction or as complex as mixing elements that exist in various microfluidic devices such as herringbone structure as shown in FIG. 10. Other mixing elements 230 that may be used herein include but are not limited to hydrodynamic flow focusing (HFF), staggered herringbone micromixer (SHM), bifurcating mixers, baffle mixers, T-junction mixing, etc. The resulting solution receptacle 240 comprises a receptacle for receiving the resulting solution 242 resulting from rapid mixing by mixing element 230.

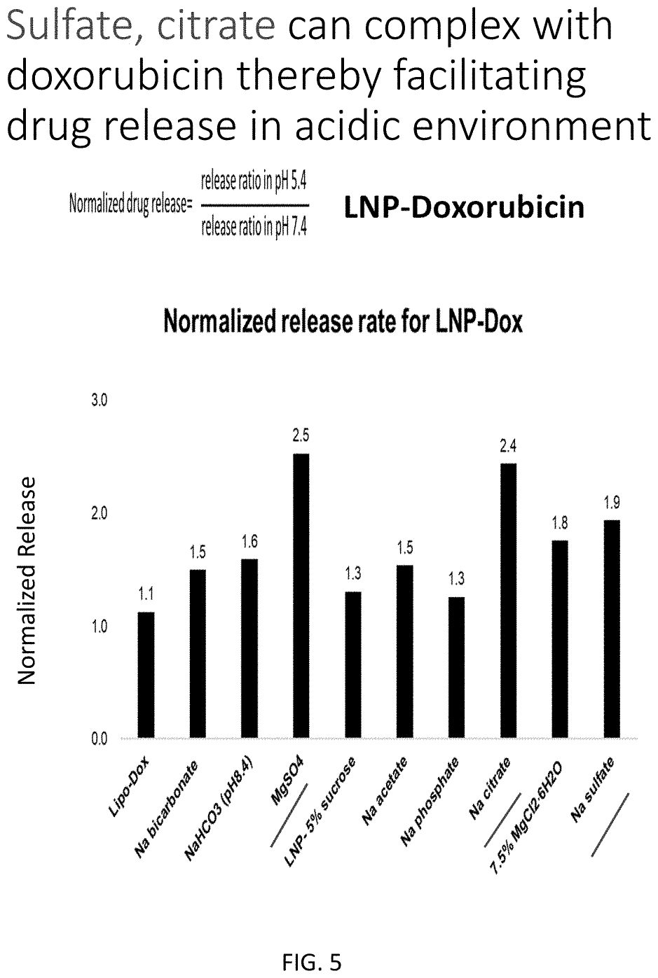

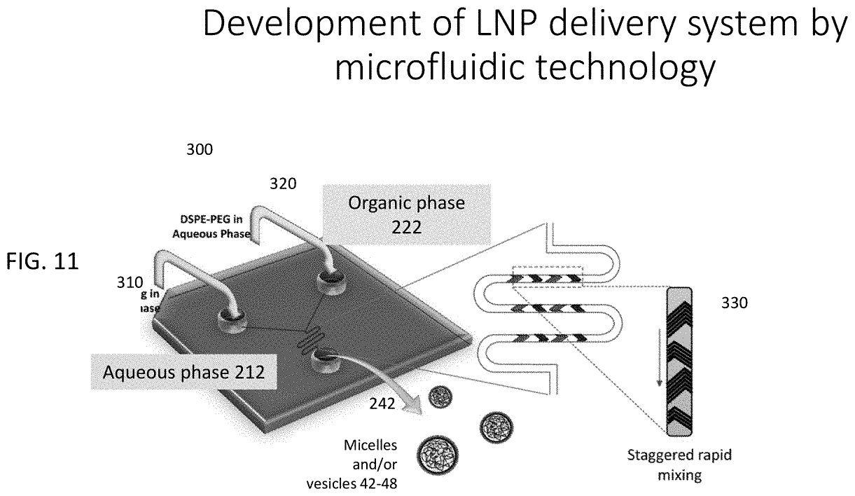

An exemplary rapid mixing system 200 comprises a microfluidic device 300 as shown in FIG. 11. In an embodiment, the rapid mixing with microfluidic device 300 with herringbone mixing element 330 may be adjusted by varying parameters such as flow rate ratio (FKR) and total flow rate (TFR) of the phases 212 and 222. FRR is defined as the ratio of flow rate of the organic phase 222 to aqueous phase, and TFR is the sum of the two flow rates. These two parameters along with other factors such as lipid composition, ratio of charged lipid, cholesterol concentration, lipid concentration, drug loading, etc. . . . can influence characteristics of the resulting LNP 1 such as LNP diameter, D/L ratio, EE % etc. . . . discussed in further detail below in connection with FIG. 5. An embodiment of the microfluidic device 300 which is used in the Examples below is based off of the microfluidic device used in Belliveau et al.⁶, which features a mixing channel 200 μm wide and 79 μm high, with herringbone structures formed by 31 μm high and 50 μm thick features on the roof of the channel Fluidic connections were made with 1/32″ I.D., 3/32″ O.D. tubing that was attached to 21G1 needles for connection with syringes. One mL or 3 mL syringes were used for inlet streams. Two syringe pumps were used to control the flow rate through the device. The syringe pump introduces the two solutions into the microfluidic device, where they come into contact at the Y-junction. ⁶ Belliveau, N. M. et al. (2012). Microfluidic Synthesis of Highly Potent Limit-size Lipid Nanoparticles for In Vivo Delivery of siRNA. Molecular Therapy—Nucleic Acids, 1, e37. https://doi.org/10.1038/mtna.2012.28

Rapid mixing may also be achieved using T or Y junction as mixing element 230 for rapid mixing, where two fluid mixing at the T junction can provide adequate rapid mixing to achieve the LNP 1 of the present invention. The flow rate for aqueous phase may be from about 1 about 1 mL/min to about 6 mL/min, such as about 1 about 1 mL/min, about 2 mL/min, about 3 mL/min, about 4 mL/min, about 5 mL/min or about 6 mL/min including all numbers ranges and numbers falling within these values. The flow rate for organic phase may be from about 2 mL/min to about 6 mL/min, such as about 2 mL/min, about 3 mL/min, about 4 mL/min, about 5 mL/min, about 6 mL/min including all numbers ranges and numbers falling within these values. The total flow rate may be from about 1 mL/min to 12 mL/min such as about 1 mL/min, about 2 mL/min, about 3 mL/min, about 4 mL/min, about 5 mL/min, about 6 mL/min, about 7 mL/min, about 8 mL/min, about 9 mL/min, about 10 mL/min, about 11 mL/min or about 12 mL/min including all numbers ranges and numbers falling within these values.

FIG. 2 illustrates an embodiment of the method of LNP preparation 100 of the present invention. As illustrated in FIG. 2, the first steps 110 and 120 involve preparation of organic phase solution 222 and preparation of aqueous phase solution 212, respectively. In an embodiment, the step 110 of preparation of the organic phase solution 222 comprises mixing the desired amount of neutral 12 and anionic 22 lipids in a solvent such as ethanol. In an embodiment, the organic phase may further comprise a combination of cholesterol and/or DSPE-PEG2000 26. In an embodiment, the step 120 of preparation of the aqueous phase solution 212 comprises mixing the desired amount of positively charged hydrophilic drug 30 such as dox, cDDP or doxy in NaCl solution. In one embodiment, the drug concentration in the aqueous phase 212 may range from about 1 mg/mL to about 20 mg/mL such as about 1 mg/mL, about 2 mg/mL, about 4 mg/mL, about 6 mg/mL, about 8 mg/mL, about 10 mg/mL, about 12 mg/mL, about 14 mg/mL, about 16 mg/mL, about 18 mg/mL, about 20 mg/mL including all numbers ranges and numbers falling within these values, and the lipid concentration in the organic phase 222 may range from about 1 mg/mL to about 40 mg/mL such as about 1 mg/mL, about 3 mg/mL, about 5 mg/mL, about 10 mg/mL, about 15 mg/mL, about 20 mg/mL, about 25 mg/mL, about 30 mg/mL, about 35 mg/mL or about 40 mg/mL including all numbers ranges and numbers falling within these values. In addition, the reaction temperature for the preparation step ranged from room temperature of about 25° C. to about 65° C.

Next in step 130, the organic phase 222 and aqueous phase 212 undergo rapid mixing. In one embodiment, the rapid mixing step 130 is performed using the rapid mixing system 200. In one embodiment, the rapid mixing step 130 is performed using a microfluidic device 300. In another embodiment, the rapid mixing step 130 is performed using a microfluidic device 300 with a herringbone structure. In yet another embodiment, the rapid mixing step 130 is performed using a microfluidic device 300 with a herringbone structure with TFR from about 10 to 14 mL/min and FRR from about 2:1 to 6:1. In another embodiment, the rapid mixing step 130 is performed using a T junction.

Next, in step 140, the resulting solution 242 is collected from the resulting solution receptacle 240. The resulting solution 242 comprises one or more of reverse spherical micelle 42, small unilaminar vesicle 44, reverse cylindrical micelle 46 and/or small multilaminar vesicle 48. Next, in step 150, dialysis is performed on resulting solution 242 with the addition of dialysis solution comprising dialysis buffer 252 to form the LNP 1 of the present invention. Specifically, the dialysis solution helps to dilute alcohol content of the resulting solution 242 to facilitate combination of the various micelles and vesicles 42-48 of the resulting solution 242. The dialysis solution also provides the counter ions 34 of the dialysis buffer 252 to complex with the API 30 and the charged lipid 22 to form the lipid-API-ion complex 30. In an embodiment, the dialysis buffer 252 comprises bivalent buffer. In another embodiment, the dialysis buffer 252 may comprise sodium chloride, sodium bicarbonate, magnesium sulfate etc. . . . as listed in FIG. 4. In an embodiment, the dialysis buffer 252 concentration in the dialysis solution approximates the concentration that exists in the human body. In an embodiment, the amount of the dialysis solution containing the dialysis buffer 252 to be added during the dialysis step is about 5-7 times the volume of the resulting solution 242. In an embodiment, the dialysis step 150 may be performed using a Tangential Filter Flow System (TFF) at about 18 ml/min to about 35 ml/min and about room temperature. An embodiment of the TFF may be the mini MAP.03 System from LEF Science of Taipei, Taiwan.

In an embodiment, the method of preparation 100 of the present invention provides all lipids necessary to form the LNP 1 of the present invention in one step 130 so that no further addition of lipids is necessary in any subsequent steps for the formation of LNP 1 of the present invention.

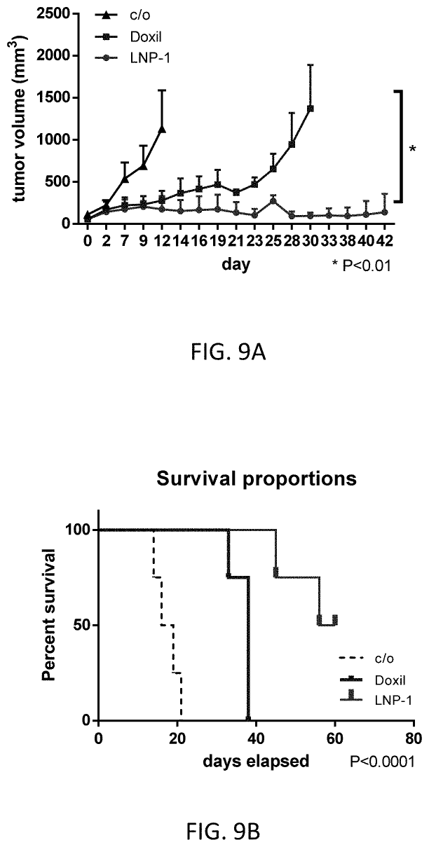

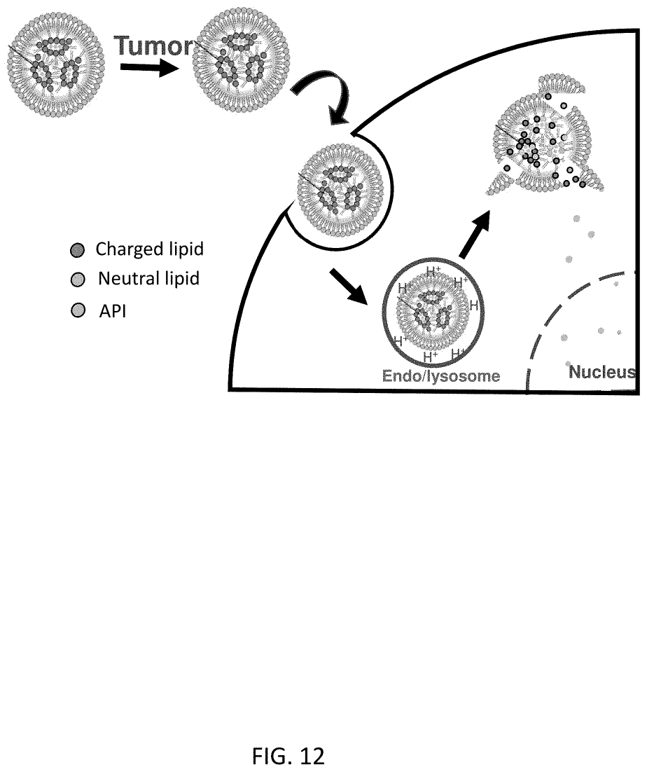

LNP 1 of the present invention may enter lysosomes of the tumor cells via endocytosis as illustrated in FIG. 12. This environment is hydrogen ion rich. These positively charged hydrogen ions create electrostatic imbalance for the LNP causing instability of the lipid-API-ion complexes 20 to break apart which, in turn, causes the LNP monolayer 10 to also break apart, releasing the API 30 into tumor cells. The combination of the anionic lipids 22 and ions 34 balancing against the positively charged API 30 allows for this electrostatic imbalance in the tumor cells to be substantially greater than if the ions 34 were not present. In this manner, the LNP 1 of the present invention substantially improves normalized release and in vivo therapeutic effects as shown in the Examples below in connection with FIGS. 9A and 9B. Finally, in step 160, LNPs 1 of the present invention are collected from TFF and stored at about 4° C. away from light.

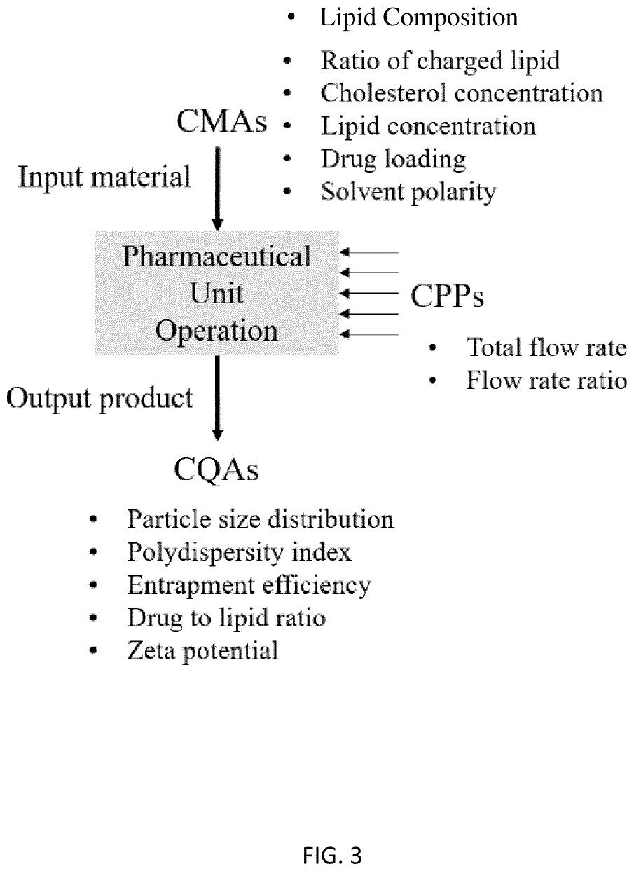

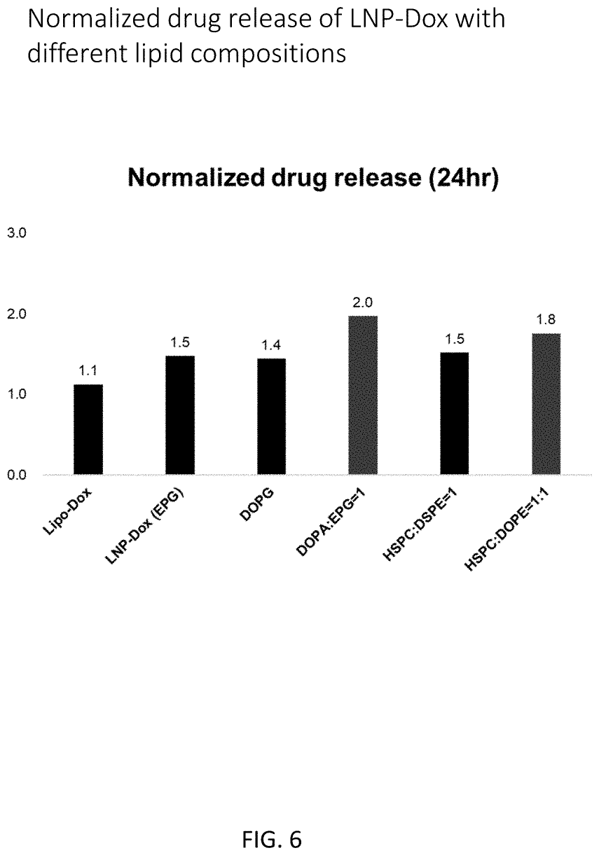

Various factors that can influence characteristics of the LNP 1 of the present invention are illustrated in FIG. 6. As illustrated in FIG. 6, ratio of charged lipid to neutral lipid, cholesterol concentration, lipid concentration, drug loading, solvent polarity as well as lipid composition are important factors in addition to TFR and FKR that influence characteristics of the resulting LNP. These LNP characteristics include particle size distribution, polydispersity index, entrapment efficiency, drug to lipid ratio and zeta potential. Furthermore, the type and even particular dialysis buffer added in step 150 of the method of preparation of the LNP 1 of the present invention substantially influences the outcome of the preparation such as overall zeta potential, normalized release, etc. . . . as discussed further below in connection with Examples and FIGS. 3-8.

In an embodiment, total lipids (weight/volume) of the organic phase 222 is about 200 mg/mL to about 1 mg/mL such as about 200 mg/mL, about 180 mg/mL, about 160 mg/mL, about 140 mg/mL, about 120 mg/mL, about 100 mg/mL, about 80 mg/mL, about 70 mg/mL, about 60 mg/mL, about 50 mg/mL, about 40 mg/mL, about 30 mg/mL, about 20 mg/mL, about 10 mg/mL, about 5 mg/mL or about 1 mg/mL including all numbers ranges and numbers falling within these values. In an embodiment, the total lipid to cholesterol ratio of the organic phase 222 ranges by weight may range from no cholesterol at all to 1:2 such as no cholesterol, about 10:1, about 7.5, about 5:1, about 2:1, about 1:1 or about 1:2 including all numbers ranges and numbers falling within these values. In an embodiment, anionic lipid to neutral lipid molar ratio is about 100:1 to about 0.1:1 such as about 100:1, about 90:1, about 80:1, about 70:1, about 60:1, about 50:1, about 40:1, about 30:1 about 20:1, about 15:1, about 7:1, about 5:1, about 3:1, about 0.75:1, about 0.5:1 or about 0.1:1 including all numbers ranges and numbers falling within these values. In an embodiment, drug loading is about 100 mg/mL to about 0.1 mg/mL such as about 100 mg/mL, about 90 mg/mL, about 80 mg/mL, about 70 mg/mL, about 60 mg/mL, about 50 mg/mL, about 40 mg/mL, about 30 mg/mL, about 20 mg/mL, about 10 mg/mL, about 8 mg/mL, about 6 mg/mL, about 4 mg/mL, about 2 mg/mL, about 1 mg/mL or about 0.1 mg/mL including all numbers ranges and numbers falling within these values. In an embodiment, TFR used in the microfluidic device 300 is about 30 to about 0.1 mL/min such as about 30 ml/min, about 25 ml/min, about 20 ml/min, about 15 ml/min, such as about 10 ml/min, such as about 5 ml/min, such as about 2 ml/min, such as about 1 ml/min, such as about 0.1 ml/min including all numbers ranges and numbers falling within these values. In an embodiment, the FRR used in the microfluidic device 300 ranges between about 10:1 to about 1:1 such as about 10:1, about 8:1, about 6:1, about 4:1, about 2:1 or about 1:1 including all numbers ranges and numbers falling within these values. In an embodiment, the organic phase may comprise both ethanol (EtOH) and isopropanol. In another embodiment, the organic phase may comprise organic solutions such as methanol, EtOH, isopropanol, or other organic solutions which can dissolve in aqueous phase.

In an embodiment, the entrapment efficiency of LNP-doxy 1 of the present invention is greater than about 20%, greater than about 30%, greater than about 40%, greater than about 50%, greater than about 60% or about 65%.

In an embodiment, the polydispersity index of the LNP 1 of the present invention is lower than 0.5, lower than 0.3, lower than 0.2 or about 0.089.

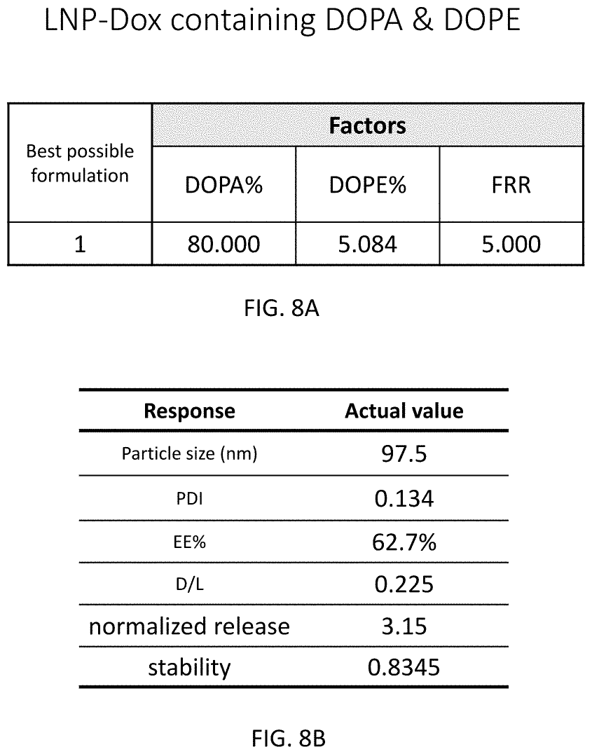

As illustrated in Example 2 below and FIGS. 6-8, different lipids compositions comprising LNP 1 of the present invention result in different LNP characteristics. Specifically, Example 2 and FIG. 6 demonstrate that LNP 1 of the present invention comprising DOPA or DOPE lipids each provided higher normalized release than commercially available lipo-dox Doxil® as well as LNP 1 of the present invention made without DOPA or DOPE lipids. Furthermore, LNP 1 of the present invention comprising both DOPA and DOPE lipids resulted in highly desirable combination of characteristics. Specifically, as illustrated in FIG. 8, an embodiment of the LNP 1 of the present invention made with both DOPA and DOPE lipids provided high normalized release at about 3.15, high EE % at about 62.7% and high DL ratio at about 0.225 while having particle size of below about 100 nm in diameter with low PID of about 0.134.

Therefore, in an embodiment, the LNP 1 of the present invention comprises DOPA lipid. In an embodiment, the LNP 1 of the present invention comprises both DOPA and DOPE lipids. In another embodiment, the organic phase 222 comprises DOPA at molar ratio of about 20 to 45 such as about 20, about 25, about 30, about 35, about 40 or about 45 including all numbers ranges and numbers falling within these values. In another embodiment, the organic phase 222 further comprises DOPE at molar ratio of about 0.1 to 1 such as about 0.1, about 0.2, about 0.4, about 0.6, about 0.8 or about 1 including all numbers ranges and numbers falling within these values. In another embodiment, the LNP 1 of the present invention is prepared with microfluidic device 300 with herringbone mixing element 330 using FRR at about 3 to about 7 such as about 3, about 4, about 5, about 6 or about 7 including all numbers ranges and numbers falling within these values.

In another embodiment, the LNP 1 of the present invention comprises EPG, HSPG lipids, DSPE-mPEG2000, 14:0 PEG 2000 PE or a combination thereof. In an embodiment, the organic phase 222 comprises molar ratio of anionic lipid 22 to neutral lipid 12 at about 15 to 1 such as about 15, about 13, about 11, about 9, about 7, about 5, about 3, about 2 or about 1 including all numbers ranges and numbers falling within these values. In an embodiment, the organic phase 222 comprises EPG and HSPG at molar ratio of about 15 to 1 such as about 15, about 13, about 11, about 9, about 7, about 5, about 3, about 2 or about 1 including all numbers ranges and numbers falling within these values. In an embodiment, the organic phase 222 comprises EPG at about 25 to 65 molar ratio such about 25, about 30, about 35, about 40, about 45, about 50, about 55, about 60 or about 65 including all numbers ranges and numbers falling within these values, HSPG at about 5-15 molar ratio such about 5, about 6, about 7, about 8, about 9, about 10, about 11 about 12, about 13 about 14 or about 15 including all numbers ranges and numbers falling within these values. In another embodiment, the organic phase 222 further comprises DSPE-mPEG2000 at about 0-4 molar ratio and/or 14:0 PEG 2000 PE at about 0.4 molar ratio. In yet another embodiment, the molar ratio of anionic lipids 22 to neutral lipids 12 in the LNP 1 of the present invention is the same as the molar ratio of organic phase 222.

The present invention also comprises method of treatment of illnesses such as cancer using any embodiments of the LNP 1 of the present invention disclosed comprising administration of the LNP 1 of the present invention to the subject.

According to the invention, the administration can be rectal, nasal, vaginal, parenteral or topical. More preferably, the administration is parenteral including but not limited to intravenous, subcutaneous, intramuscular, intradermal and intraperitoneal. More preferably, the administration is intravenous. Preferably, the targets are tumors. Preferably, the drug is an anti-tumor drug.

According to the invention, after administering LNP 1 of the present invention to a subject, the LNPs accumulate at the tumor sites due to enhanced permeability and retention effect (EPR). Specifically, EPR results from the fact that tumors tend to be nutrient and oxygen rich due to fast growing blood vessels in tumors. However, those fast growing blood vessels comprise irregularly positioned endothelia cells resulting in gaps that allow nanoparticles of 200 nm or less such as the LNP 1 of the present invention to escape blood vessels. Combining this with the fact that tumors lack lymphatic systems that can flush away nanoparticles, EPR effects result in LNPs 1 of the present invention accumulating at the tumor.

We claim the therapy of subject, e.g., mammals such as mice, rats, simians, and human patients, with cancers including, but not limited to breast, prostate, colon, non-small lung, pancreatic, testicular, ovarian, cervical carcinomas, head and neck squamous cell carcinomas.

As used herein, “administration, delivered or administered” is intended to include any method which ultimately provides the drug/LNP complex to the tumor mass. Examples include, but are not limited to, topical application, intravenous administration, parenteral administration or by subcutaneous injection around the tumor. Tumor measurements to determine reduction of tumor size are made in two dimensions using vernier calipers twice a week.

For in vivo administration, the pharmaceutical compositions are preferably administered parenterally, i.e., intravenously, intraperitoneally, subcutaneously, intrathecally, injection to the spinal cord, intramuscularly, intraarticularly, portal vein injection, or intratumorally. More preferably, the pharmaceutical compositions are administered intravenously or intratumorally by a bolus injection.

Administration in vivo can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents can be empirically determined by those of skill in the art.

It should be noted that all preferred embodiments discussed for one or several aspects of the invention also relate to all other aspects. This particularly refers to the amount and type of neutral and/or anionic lipid, the amount and type of active agent, the amount and type of further active agent for combination therapy, and the type of disorder to be treated.

The following examples should be illustrative only but are not meant to be limiting to the scope of the invention. Other generic and specific configurations will be apparent to those skilled in the art.

### EXAMPLES

LNP 1 of the present invention used in Examples were prepared using the method of preparation 100 of the present invention. For Example 1 and 3, the organic phase 222 for preparing the LNP 1 of the present invention comprises EPG at about 46.8 molar ratio, HPSC at about 9.4 molar ratio, cholesterol at about 41.9 molar ratio and DSPE-mPEG2000 at about 1.9 molar ratio dissolved in EtOH at total lipid concentration about 18.6 mg/mL. The aqueous phase 212 for preparing the LNP 1 of the present invention comprises dox in NaCl at concentration of 0.9%. The organic and aqueous phases are then injected into a microfluidic device 300 with herringbone mixing structure as described above in connection with FIG. 4 at microfluidic device parameters TFR about 12 mL/min and FRR about 4:1. Dialysis step 150 is then performed on the resulting solution 242 to form and isolate the LNP 1 of the present invention. For Example 1, different dialysis buffers 252 were used in the dialysis solutions at physiological concentration to result in LNP 1 of the present invention with different characteristics, including NaCl, CaCl₂, MgSO₄ as shown in FIGS. 4-5. For Example 3, MgSO₄ dialysis buffer 252 was used in the dialysis solution at physiological concentration to result in LNP 1 of the present invention.

The Examples use the following method to determine concentration of doxorubicin: First, 10 mg/mL dox was diluted in 95% methanol to various concentrations at 10, 5, 2.5, 1.25, 0.625, 0.3125 g/mL to serve as calibration solutions. The dox sample in question requiring concentration measurement is diluted with 95% methanol to 100 times by volume. Then, UV-Visible spectrophotometer at 470 nm wavelength light was used to determine dox concentration by interpolation after comparison with the calibrating solutions. All processes were conducted at room temperature of about 25° C.

### Example 1—In Vitro Normalized Release of LNP 1 of the Present Invention Encapsulating Dox Prepared Using Various Dialysis Buffers in Step 130

During experiments, PBS buffer was used to simulate a physiological environment at pH of 7.4, while citrate buffer was used to simulate a tumor microenvironment at pH of 5.4, wherein the citrate buffer contains sodium citrate dehydrate and citric acid and its pH can be adjusted using NaOH and HCl. These buffers are not to be confused with dialysis buffers 252 of step 130 as they have no role in complexing with API nor formation of the LNP 1 of the present invention. The in vitro API release was evaluated in these two different solutions using the following steps:

1 mg/mL LNP-dox solution in PBS buffer was prepared by diluting LNP-dox using PBS buffer. The solution was placed into a 1 mL/5 cm dialysis tube (Float-A-lyzer), in which the pore size is 100 kDa. The tube was placed into a beaker containing 150 mL dialysis buffer. The beaker was then placed into a 37° C. water bath. Samples of 1 mL solution were taken from the beaker at 0, 0.5, 1, 2, 4, 8, 26, and 31 hour(s). The API content in the samples was analyzed using UV-Vis spectrophotometer such as the Spectra/Por float-A-lyer G2. Following each sampling process, 1 mL of dialysis solution from the tube was added to the beaker to maintain consistent volume. Three forms of the API dox were used, including free form dox, LNP-dox of the present invention, and Lipo-dox (Doxil®).

1 mg/mL LNP-dox solution in citrate buffer was prepared by performing the aforementioned steps but replacing PBS buffer with citrate buffer.

FIG. 5 illustrates result of the experiment. As show in FIG. 5, each LNP 1 of the present invention has higher normalized release than Lipo-Dox. In particular, LNP 1 of the present invention has substantially higher normalized release at about 2 or above when prepared with dialysis buffers 252 with citrate or sulfate counter ions 34.

### Example 2—Effect of Lipid Composition on LNP Characteristics

Various LNP 1 encapsulating dox of the present invention were each made with different lipid compositions to explore effect of the different lipid or lipid combinations on LNP characteristics such as normalized drug release as well as particle size and PDI. The lipid compositions each comprises various combinations of one or more of EPG, DOPA, HSPC, DOPE, DOPG, DSPE-mPEG2000 and/or 14:0 PEG2000 PE lipid composition.

Five LNP 1 containing Dox of the present invention were made using various combinations of EPG, DOPA, DOPG, DOPE and DSPE lipids, using the method described above in connection with FIG. 2. Specifically, the formulations in molar ratio are presented in Table 1:

TABLE 1
LNP Formulations in Molar Ratio of Organic Phase 222

| | | | | | | | DSPE- | 14:0 PEG2000 |
|---|---|---|---|---|---|---|---|---|
| LNP | EPG | A:DOPA | HSPC | B:DOPE | DOPG | Cholesterol | mPEG2000 | PE |
| LNP-Dox | 46.8 | 0 | 9.4 | 0 | 0 | 41.9 | 1.9 | 0 |
| DOPA:EPG | 23.4 | 23.4 | 9.4 | 0 | 0 | 41.9 | 1.9 | 0 |
| DOPG | 0 | 0 | 9.4 | 0 | 46.8 | 41.9 | 1.9 | 0 |
| HSPC:DOPE | 46.8 | 0 | 4.7 | 4.7 | 0 | 41.9 | 1.9 | 0 |
| HSPC:DSPE | 46.8 | 0 | 9.4 | 0 | 0 | 41.9 | 0 | 1.9 |

Dox release in pH 7.4 PBS buffer was determined using the method described above. DOPA LNP released the least amount of Dox after 24 hours followed by DOPE LNP compared to LNP 1 of the present invention made with other lipids indicating that DOPA and DOPE LNP resulted in the highest stability at or near neutral pH 7.4.

Next, dox release in citrate buffer at pH 5.4 were measured for each of the LNP at 24 hours in order to determine normalized release with results shown in FIG. 6. As illustrated in FIG. 6, DOPA and DOPE LNPs (DOPA DOPE LNP) provided the two highest normalized release by substantial margin over LNP made with other lipids as well as Doxil®.

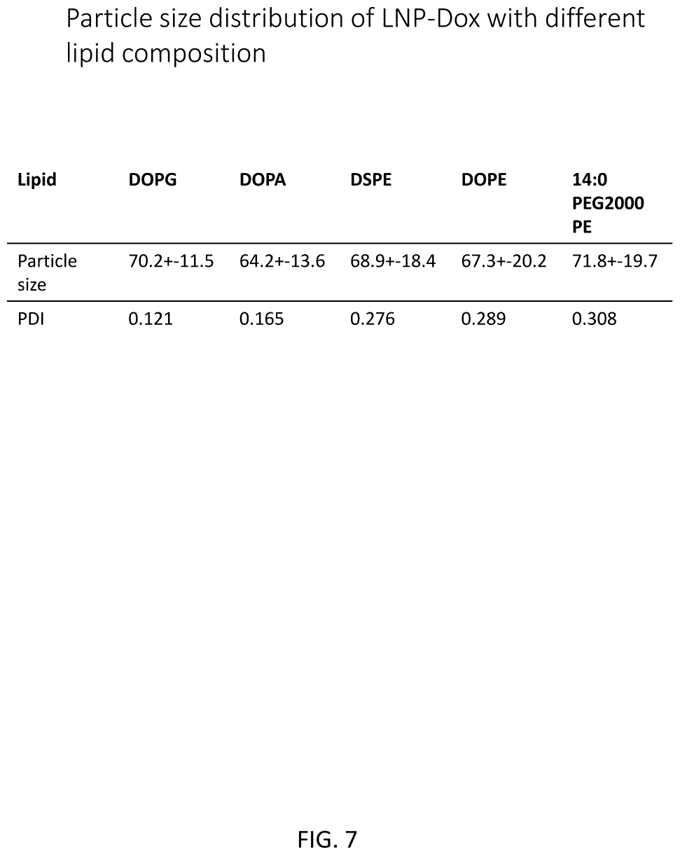

FIGS. 6 and 7 illustrate physical characteristics of the LNP 1 of the present invention made with the various lipid compositions listed in Table 1. As shown in FIG. 7, DOPA LNP provided the smallest particle size followed by DOPE LNP, however, DOPA LNP provided substantially smaller PDI. These experiments indicate that DOPA and DOPE lipids both provided desirable characteristics for making LNP 1 of the present invention.

Next, LNP 1 of the present invention was made using a mixture of DOPA and DOPE lipids (DOPA DOPE LNP 1 of the present invention), using the formulation illustrated in FIG. 8A. FIG. 8B illustrates the characteristics of the DOPA DOPE LNP 1 of the present invention. As shown in FIG. 8B, normalized release is at 3.15, which is nearly 3 times the normalized release of commercially available Doxil® and substantially higher than any LNP shown in FIG. 6 while providing a DL ratio nearly twice that of the commercial product Lipo-dox at a low PDI of 0.134 and particle size still below 100 nm. Therefore, a mixture of DOPA and DOPE lipids provided surprising high normalized release indicating the ability of DOPA DOPE LNP 1 of the present invention to specifically target tumor sites.

### Example 3—In Vivo Therapeutic Efficacy of LNP of the Present Invention Encapsulating Dox

C26 human cancer cell line was implanted into the backs of BALB/c mice to create colon-26 carcinoma tumor-bearing mice. When the tumor grew to 100 mm³, LNP 1 of the present invention containing dox was injected via mice's vein located in the tail at a dosage of 5 mg/kg of dox, once per week. At predetermined time periods as shown in FIG. 9A, tumor volume was determined by Vernier caliper. The experiment continued until either mouse death or the tumor size exceeding 1,000 mm³.

FIGS. 9A and 9B illustrate results of the experiment. As shown in FIG. 9A, both Lipo-dox and the LNP 1 of the present invention resulted in substantially lower tumor volume than control and free form dox. In fact, LNP 1 of the present invention controlled tumor volume well below 500 mm³ up to 42 days whereas Lipo-dox exceeded 1000 mm³ by day 28.

With regards to the survival curve of FIG. 9B, the LNP 1 of the present invention only resulted in 50% death by day 60. In comparison, 100% of lipo-dox treated mice died by day 40. 100% of the mice in control group died. The trials demonstrate that LNP of the present invention provide substantially superior therapeutic effects than Lipo-dox and free form dox while providing better protection than free form dox from toxicity.

What is claimed is:

1. A lipid-based nanoparticle (LNP) comprising an outer lipid monolayer encapsulating a plurality of lipid-active pharmaceutical ingredient-ion (lipid-API-ion) complexes
wherein each lipid-API-ion complex comprises a complex of charged lipid, active pharmaceutical ingredient (API) and ion;
wherein the outer lipid monolayer of the LNP comprises neutral lipids;
wherein the charged lipid of the lipid-API-ion complex comprises anionic lipids;
wherein the API is cationic; and
wherein the ion is anionic.

2. The LNP of claim 1, wherein the LNP comprises a combination of 1,2-dioeoyl-sn-glycero-3-phosphate (DOPA) and 1,2-dioleoylphosphoethanolamine (DOPE) lipids.

3. The LNP of claim 2, wherein the molar ratio of DOPA to DOPE lipids is from about 400 to about 33.

4. The LNP of claim 3, wherein the LNP further comprises cholesterol at ratio by weight of total lipid to cholesterol ranging from about 10:1 to about 1:2.

5. The LNP of claim 1, wherein the LNP comprises anionic to neutral lipids at a molar ratio of about 12 to about 2.5.

6. The LNP of claim 5, wherein the anionic lipid comprises egg L-α-phosphatidylglycerol (EPG) and neutral lipid comprises L-α-phosphatidylcholine, hydrogenated soybean phosphatidylcholine (HSPC).

7. The LNP of claim 1, wherein the API is a positively charged form of doxorubicin or a pharmaceutically acceptable salt form thereof.

8. The LNP of claim 7, wherein the drug to lipid ratio is greater than about 0.15.

9. The LNP of claim 7, wherein the normalized release is greater than about 1.2, about 1.3, about 1.4 or about 2.0.

10. The LNP of claim 1, wherein the positively charged API is a positively charged form of doxycycline or a pharmaceutically acceptable salt form thereof.

11. The LNP of claim 10, wherein the drug to lipid ratio is greater than about 0.17.

12. The LNP of claim 1, wherein the positively charged API is a positively charged form of cisplatin or a pharmaceutically acceptable salt form thereof.

13. The LNP of claim 12, wherein the drug to lipid ratio is greater than about 0.12.

14. The LNP of claim 1 made using rapid mixing.

15. The LNP of claim 14, wherein the rapid mixing is performed using microfluidic device with staggered herringbone fluid mixing element.

16. The LNP of claim 1, wherein the LNP does not comprise a therapeutically effective amount of nucleic acids, nucleotides or polynucleotides.

17. The LNP of claim 1, wherein the ion comprises bivalent ion.

18. The LNP of claim 1, wherein the LNP does not comprise any phosphatidyl glycerol lipids (PGL) or PGL derivatives.

19. A method of preparation for the lipid-based nanoparticle LNP of claim 1 comprising the steps of
a. Prepare organic phase comprising neutral and anionic lipids.
b. Prepare aqueous phase comprising a positively charged form of an active pharmaceutical ingredient (API).
c. Rapid mix the organic and the aqueous phases to make a resulting solution.
d. Perform dialysis on the resulting solution using dialysis solution comprising dialysis buffer to form and isolate the LNP of the present invention.

20. The method of preparation of claim 19, wherein all necessary lipids for forming the LNP of claim 1 is in the organic phase of the prepare organic phase step.

21. The method of preparation of claim 19, wherein the API comprises a positively charged form of doxorubicin, doxycycline or cisplatin or a pharmaceutically acceptable salt form thereof.

22. The method of preparation of claim 19, wherein the rapid mixing step is performed using a rapid mixing system comprising an organic phase pump, an aqueous phase pump and a mixing element.

23. The method of preparation of claim 22, wherein the mixing element comprises a T junction or a Y junction.

24. The method of preparation of claim 22, wherein the mixing element comprises a microfluidic device with herringbone mixing structure.

25. The LNP of claim 19, wherein the organic phase comprises a combination of 1,2-dioeoyl-sn-glycero-3-phosphate (DOPA) and 1,2-dioleoylphosphoethanolamine (DOPE) lipids.

26. The LNP of claim 25, wherein the molar ratio of DOPA to DOPE lipids is about 400 to about 33.

27. The LNP of claim 19, wherein the organic phase further comprises cholesterol at ratio by weight of total lipid to cholesterol ranging from about 10:1 to about 1:2.

28. The LNP of claim 19, wherein the organic phase comprises anionic to neutral lipids at a molar ratio of about 12 to about 2.5.

29. The LNP of claim 28, wherein anionic lipid comprises egg L-α-phosphatidylglycerol (EPG) and neutral lipid comprises L-α-phosphatidylcholine, hydrogenated soybean phosphatidylcholine (HSPC).

30. The method of preparation of claim 19, wherein the prepared LNP encapsulating doxorubicin or a pharmaceutically acceptable salt form thereof has drug to lipid ratio of greater than about 0.15.

31. The method of preparation of claim 19, wherein the prepared LNP encapsulating doxycycline or a pharmaceutically acceptable salt form thereof has drug to lipid ratio of greater than about 0.17.

32. The method of preparation of claim 19, wherein the prepared LNP encapsulating cisplatin or a pharmaceutically acceptable salt form thereof has drug to lipid ratio of greater than about 0.12.

33. The method of preparation of claim 19, wherein the dialysis step results in complexing of counter ions of the dialysis buffer with the API.

34. The method of preparation of claim 33, wherein the counter ions of the dialysis buffer comprise bivalent ions.

35. The method of preparation of claim 33, wherein the resulting solution comprises one or more of reverse spherical micelle, small unilaminar vesicle, reverse cylindrical micelle and/or small multilaminar vesicle.

36. The method of preparation of claim 35, wherein the dialysis step results in combination of the various micelles and vesicles to facilitate formation of the LNP of the present invention of claim 1.

37. A method of treatment using the LNP of claim 1 comprising the step of injecting the LNP of claim 1 into a subject at dosage of about 0.01 mg/mL to 50 mg/mL.
